(19) United States
(12) Patent Application Publication
(10) Pub. No.: US 2026/0335525 A1
(43) Pub. Date: Oct. 29, 2026

(54) BLOOD TREATMENT DEVICE FOR GAS EXCHANGE

(71) Applicant: FRESENIUS MEDICAL CARE DEUTSCHLAND GMBH, Bad Homburg (DE)

(72) Inventor: Jürgen KLEWINGHAUS, Bad Homburg (DE)

(73) Assignee: FRESENIUS MEDICAL CARE DEUTSCHLAND GMBH, Bad Homburg (DE)

(21) Appl. No.: 19/150,924

(22) PCT Filed: Jan. 19, 2024
(86) PCT No.: PCT/EP2024/051218

§ 371 Date: Jul. 25, 2025

(30) Foreign Application Priority Data
Jan. 30, 2023 (DE) 10 2023 102 145.1

(51) Int. Cl. A61M 1/36; A61M 1/16; A61M 16/00

(52) U.S. Cl. CPC A61M 1/3663; A61M 1/1629; A61M 1/1698; A61M 1/3603; A61M 16/0003; A61M 16/022; A61M 2016/0033; A61M 2202/0007; A61M 2202/0021; A61M 2202/0208; A61M 2202/0413; A61M 2205/3303; A61M 2205/3327; A61M 2205/3334; A61M 2205/3368; A61M 2205/36; A61M 2205/505; A61M 2230/005; A61M 2230/432; A61M 2230/50

(57) ABSTRACT

The invention relates to a blood treatment appliance 100 for gas exchange via a gas exchange unit 300 having two regions separated by a membrane, wherein one region has blood flowing through it and the other region has sweep gas flowing through it, and wherein the blood treatment appliance 100 comprises a pump device 101 for blood, sensors for measuring at least two treatment parameters, a device 404 for changing at least one sweep gas parameter, and a control unit 500 which is connected to the pump device 101 for blood, to the sensors for measuring at least two treatment parameters and to the device 404 for changing at least one sweep gas parameter and is configured in such a way that it controls the device 404 for changing at least one sweep gas parameter in accordance with the at least two treatment parameters. The invention further relates to a method for controlling the device 404 for changing at least one sweep gas parameter and/or for controlling a mechanical ventilation unit 600 in the blood treatment appliance 100 according to the invention.

### TECHNICAL FIELD

The present invention relates to a blood treatment appliance according to claim 1 for gas exchange via a gas exchange unit, and to a method according to claim 12 for controlling the device for changing at least one sweep gas parameter.

### BACKGROUND

The central role of the lungs is to supply the body with oxygen (O2) and to remove from the body the carbon dioxide (CO2) that arises as a result of the metabolism. During inhalation, oxygen-containing air flows into the lungs. The oxygen diffuses across the lungs into the blood and circulates through the body in order to supply the organs. The CO2 arising as a result of the metabolism is transported by the blood to the lungs, where it is eliminated from the body by exhalation. By virtue of the breathing movements and the circulation of blood, there is a continuous exchange of O2 and CO2 between the ambient air, the lungs and the blood.

If the lungs are damaged such that they can no longer sufficiently perform their gas exchange function, medical measures are required that replace or support the supply of O2 to the body and/or the removal of CO2 from the latter.

Extracorporeal lung support has become increasingly important in recent years, not least because of the Covid 19 pandemic, but also because of the increasing incidence of chronic obstructive pulmonary disease (COPD).

In patients with hypercapnia, for example in those suffering from COPD, the removal of CO2 in particular is disrupted. To be able to remove CO2 effectively from the three compartments of the body, i.e. blood, tissue and bone, these patients are generally ventilated by mask at a high gas pressure. However, since this treatment greatly restricts the patient and is found to be uncomfortable, it is generally only poorly tolerated.

In patients with hypercapnia, mechanical ventilation, which requires intubation for introducing gas into the lungs at high pressure, is as far as possible avoided, on the one hand because the CO2 may not be sufficiently removed in some cases, and, on the other hand, because the ventilation considerably restricts the patient, on account of the required sedation, and is associated with side effects and sequelae which in particular affect the lung tissue.

A further possibility available for treating hypercapnic patients is extracorporeal lung support (ECLS). In this method, blood is conveyed in an extracorporeal circuit through a gas exchange appliance, for example a membrane oxygenator or a dialyser. The gas exchange appliance takes over the gas exchange function of the lungs. The basic set-up and function of a membrane oxygenator and of a dialyser are known from the prior art. Ambient air, pure oxygen or a mixture of air with oxygen, nitrogen and/or noble gases is used as sweep gas. The proportion of the individual gas constituents of the sweep gas mixture may vary.

The level of the blood flow rate in ECLS treatment determines whether mainly oxygen is added (extracorporeal membrane oxygenation (ECMO)) and at the same time carbon dioxide removed or mainly only carbon dioxide is removed (extracorporeal CO2 removal (ECCO2R)). High blood flow rates of more than 1.5 l/min are normally required for ECMO. However, for the removal of excess CO2(ECCO2R), a low blood flow is sufficient, which can be less than 1.5 l/min or even less than 500 ml/min, and therefore treatment can be carried out with a smaller catheter for the vascular access than in ECMO, for example as is also used for dialysis (e.g. Shaldon catheter, 11-13.5 Fr). The amount of anticoagulant used, for example heparin or citrate, is also less in this treatment. Advantageously, with ECCO2R treatment, the hypercapnic patient can be treated minimally invasively and yet effectively.

However, in all extracorporeal blood treatment systems and in particular also in lung support procedures, the blood necessarily loses heat through heat radiation and heat conduction across the surface of the blood hose system and of the housing of the treatment unit and in particular also because of heat flow across the gas exchange membrane of the treatment unit. This leads to an undesired reduction of the body temperature of the patient.

Therefore, most oxygenators are equipped with a heat exchanger which is supplied with temperature-controlled water, for example from an external heating/cooling unit, and in this way ensures that the blood temperature remains substantially constant during the treatment.

However, for reasons of cost and space, appliances for ECCO2R treatment are generally not equipped with a heat exchanger or with an alternative solution for temperature control. Although the blood flow rate in ECCO2R treatment is in fact lower than in ECMO treatment, there is a not inconsiderable loss of heat even at low blood flow rates.

Furthermore, in ECCO 2R treatment, the ratio of the sweep gas flow to the blood flow, at ca. 15:1 or 5:1, is substantially greater than in ECMO treatment, which is why there are proportionately even greater heat losses. There is therefore a need to actively influence or compensate the blood heat loss during ECCO2R treatment too.

Therefore, during treatment, additional measures, for example the use of heat blankets, have to be taken in order to counteract cooling of the patient. However, these measures are often found inconvenient by the patient and are also time-consuming and interrupt the operation of the hospital.

Further factors are known from the prior art to influence the heat loss of the blood, for example the flow rate, the temperature and the humidity of the sweep gas used during ECLS treatment.

As regards the flow rate factor, it is obvious to a person skilled in the art that the heat loss rises as the flow of sweep gas increases.

It is also conceivable that, when cold sweep gas is used, a stronger heat exchange between the warmer blood and the colder sweep gas takes place than is the case where a heated sweep gas is used, since various mechanisms of heat transfer ensure that there will always be an exchange of heat between a cold medium and a warm medium. Therefore, in the case of a sweep gas heated to blood temperature, there may be assumed to be no exchange of heat between blood and sweep gas during treatment.

It is also plausible that dry sweep gas draws more heat from the blood than does humid sweep gas since, on account of the vapour pressure difference as driving force in the gas exchange, water in the form of vapour transfers across the membrane from the region of the gas exchanger through which blood flows to the region of the gas exchanger through which sweep gas flows. The water vapour condenses on the other side of the membrane and is taken up by the passing sweep gas. Through the development of “evaporative cooling”, this process leads to heat loss.

Measures which influence the abovementioned factors are already known from the prior art.

For example, the European patent EP 2 736 557 B1, which deals with the electronically controlled mixing of different sweep gas components, discloses, in addition to a heating element for heating the sweep gas, also a unit for humidifying the sweep gas in order to prevent cooling of the patient.

The U.S. Pat. No. 3,927,981 describes a blood oxygenator for O2 enrichment and removal of CO2. To reduce the gas and energy consumption, the sweep gas is heated and humidified.

Overall, it may be said that the heat loss occurring during ECLS treatment may be reduced by decreasing the flow of sweep gas or by heating or humidifying the sweep gas.

It has however been found that, when using the abovementioned measures, the gas exchange itself is less effective, for example because less CO2 is removed from the blood. Consequently, either the frequency of treatment or the duration of treatment, or both, must be increased, which in turn leads to greater cooling of the patient.

The object of the present invention is therefore to overcome this disadvantage by proposing an improved blood treatment appliance which allows the blood treatment to be optimized in such a way that the patient experiences the least possible drop in temperature but still receives the most effective treatment possible, regardless of whether O2 is added and/or CO2 removed.

The object according to the invention can be achieved by a blood treatment appliance having the features of claim 1 and by a method having the features of claim 12. Moreover, the dependent claims each contain advantageous embodiments of the invention.

### SUMMARY OF THE INVENTION

The blood treatment appliance according to the invention for gas exchange via a gas exchange unit comprises a pump device for blood, sensors for measuring at least two treatment parameters, and a device for changing at least one sweep gas parameter.

The blood treatment appliance further comprises a control unit which is connected to the pump device for blood, to the sensors for measuring at least two treatment parameters and to the device for changing at least one sweep gas parameter. The control unit is configured in such a way that it controls the device for changing at least one sweep gas parameter in accordance with the at least two treatment parameters. The control unit can serve purely as a control device or as a regulating device and execute all or substantially all of the method steps.

The term gas exchange encompasses the removal of CO2 from the extracorporeal blood circuit or the supply of O2 into the extracorporeal blood circuit, or both.

The gas exchange unit is composed of a housing that contains hollow fibre bundles. The hollow fibre bundles form a semipermeable membrane which separates the gas exchange unit into two regions. Therefore, at least in countercurrent processes, the blood flows for example inside the hollow fibres and the sweep gas outside the hollow fibres, or vice versa.

The gas exchange unit can be a commercially available membrane oxygenator or dialyser. The hollow fibre bundles of the gas exchange unit are preferably composed of a hydrophilic microporous hollow fibre, for example of polysulfone or polyethersulfone, containing a fraction of polyvinylpyrrolidone (PVP). The hollow fibres can also be composed of polymethylpentene (PMP), if the geometry allows humidification without the membrane being closed by an accumulation of very small water droplets.

The term treatment parameter encompasses, for example, the patient's temperature, the carbon dioxide partial pressure (pCO2) or the blood flow rate. The patient's temperature can be measured by means of a suitable sensor either in the blood line or directly on the patient or can also be read into the hospital data system via a data interface. The pCO2, which reflects the amount of the CO2 dissolved in the arterial blood, can be measured in the sweep gas by means of a suitable sensor, preferably downstream of the gas exchange unit, or in the blood, for example by means of a blood gas analysis. The blood flow rate can be measured via a sensor integrated in the pump device for blood, or via a flow sensor located in the arterial or venous line in the extracorporeal blood circuit.

The sweep gas parameters comprise the temperature, the humidity or the flow rate of the sweep gas. These parameters too can be measured via suitable sensors located in the sweep gas line, there advantageously upstream of the gas exchanger.

The device for changing at least one sweep gas parameter can be a heater for heating the sweep gas, a humidifier for humidifying the sweep gas or a pump device which conveys the sweep gas along the sweep gas line. The pump device can be composed of one or more active elements for generating a flow, for example a pump. However, it can also be a passive element, or can be connected to a passive element, which sets or adjusts the flow, for example a throttle or a valve. In some embodiments, the control unit is connected to a user input module (user interface) for inputting and/or storing at least one treatment goal. Therefore, taking into account the contrary goals of minimum heat loss and maximum CO2 removal, the user can select an individual treatment goal and/or influence the course of treatment through the input of parameters.

The user input module can moreover be connected to a decision support system, which suggests parameters for the user to input that are appropriate to a situation. Alternatively or in addition, parameters from a data storage system can be used, for example parameters originating from previous treatments.

Alternatively or in addition, the use of a script is conceivable, i.e. a source text which contains a list of commands and, based on the detected values for the patient's temperature and the pCO2,automatically specified a treatment mode with a minimum heat loss and a maximum CO2 removal.

In some embodiments, the blood treatment appliance is connected to the patient and the gas exchange unit via a hose set. It can also be connected to at least one further blood treatment unit with which the blood can be acted upon in a common extracorporeal blood circuit. The action can be a mechanical, chemical, physical or other action. Such a combination therapy is used especially when the treatment techniques have advantageous synergies. The further blood treatment unit can be, for example, a dialyser for renal replacement therapy, an adsorber cartridge for therapeutic apheresis, or also a diagnostic unit, which can determine various parameters of the blood, for establishing pathological changes of the blood. In this way, ECMO treatment or ECCO2R treatment can be combined at the same time with a dialysis treatment and/or adsorption treatment. The treatment units can be combined in any desired sequence, for example as a serial arrangement.

In a further embodiment, the blood treatment appliance can comprise, in addition to the gas exchange unit, a mechanical ventilation unit, which supports the exchange of O2 and CO2. Mechanical ventilation can either take place non-invasively via a mask or invasively via an endotracheal tube, which is inserted through the nose or mouth, or via a tracheal cannula, which is inserted through an opening into the trachea. The control unit is configured in such a way that it also controls the mechanical ventilation unit in accordance with the at least two treatment parameters.

The method according to the invention can be executed largely or completely by the control unit. In particular, the control unit can execute those steps which do not require or concern human assistance and/or intervention.

In the method for controlling the device for changing at least one sweep gas parameter and/or for controlling the mechanical ventilation unit, the treatment parameters, i.e. the patient's temperature and/or the pCO2 and/or the blood flow rate, are first of all detected by the sensors and transmitted to the control unit. One or more treatment parameters can be input or preset via the user interface.

The control unit compares the detected treatment parameters (actual values) against values (setpoint values) input by the user and/or preset and calculates manipulated variables for configuring the device for changing at least one treatment parameter.

Based on the calculated values, the control unit controls the heater, humidifier and/or pump device such that the sweep gas is heated and/or humidified and/or the flow rate of the sweep gas is increased or lowered.

It is moreover conceivable to use the detected pCO2 to calculate and/or balance the total quantity of the CO2 removed. Assuming that the CO2 content at the inlet of the sweep gas is equal to or almost equal to zero, the CO2 content in percent is multiplied by the flow rate of the sweep gas.

The present invention is explained below by way of example, with reference to the accompanying drawings in which identical reference signs designate the same or similar components. In the drawings:

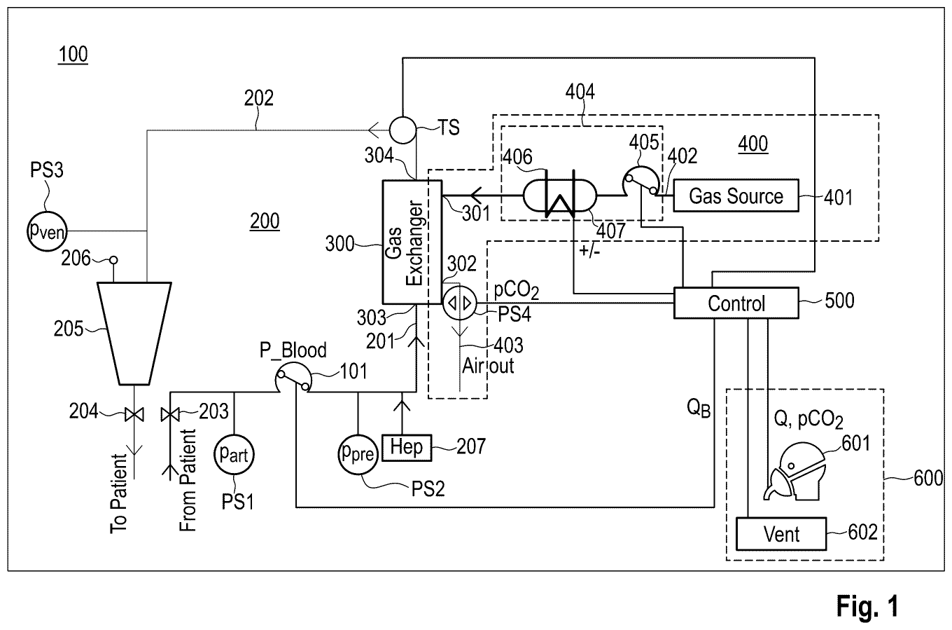

FIG. 1 shows a simplified process diagram for the blood treatment appliance according to the invention,

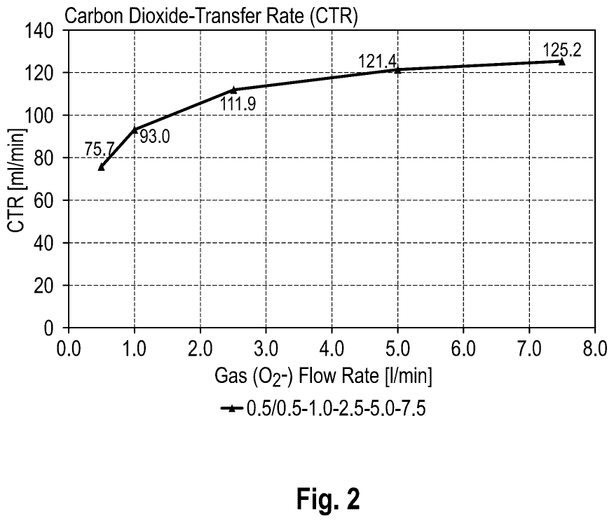

FIG. 2 shows a CO2 removal curve in accordance with the sweep gas flow,

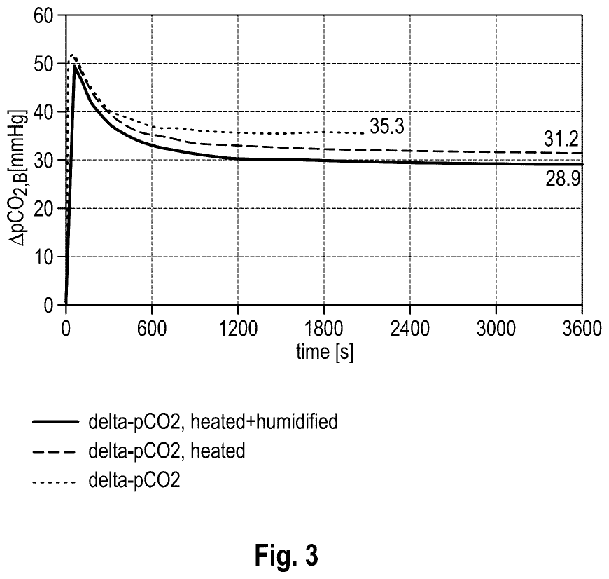

FIG. 3 shows the carbon dioxide partial pressure under different conditions of the sweep gas over time,

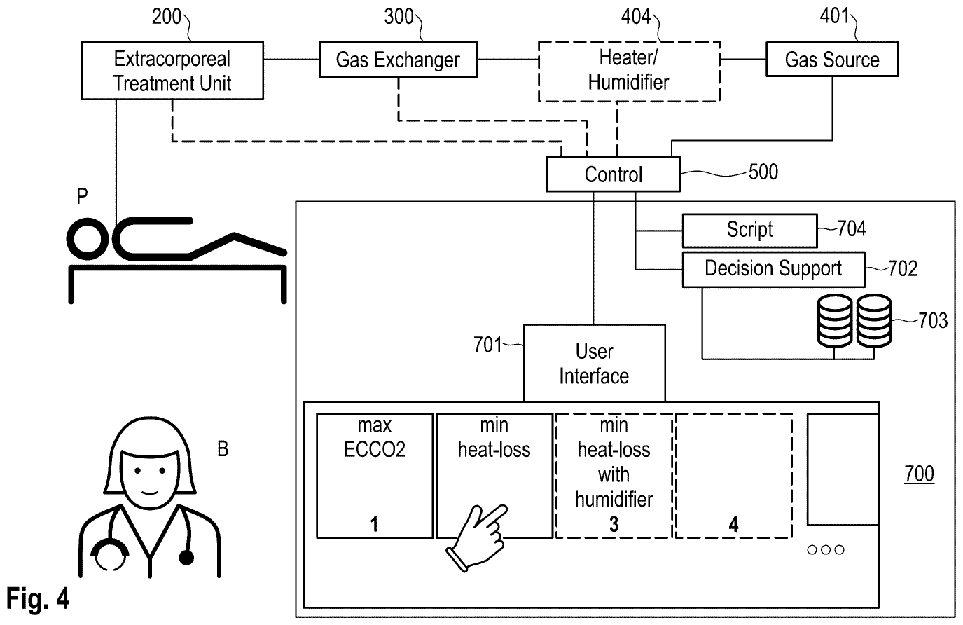

FIG. 4 shows the treatment appliance according to the invention in a schematic view,

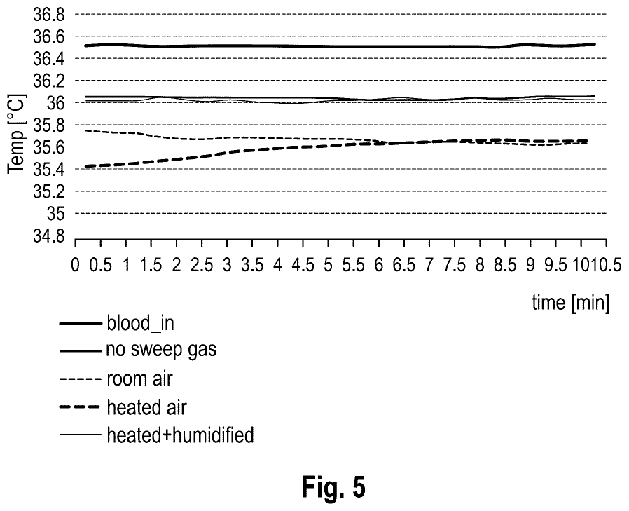

FIG. 5 shows the blood temperature under different conditions of the sweep gas.

FIG. 1 shows a simplified process diagram for the blood treatment appliance according to the invention. The blood treatment appliance 100 is optionally connected to an extracorporeal blood circuit 200 in the form of a hose set which, in an arterial blood line 201, leads towards gas exchange unit 300 and, in a venous blood line 202, leads away from the gas exchange unit 300. Both blood lines can be connected to the vascular system of a patient (not shown here). The small arrow heads indicate the direction of flow. For reasons of hygiene, the hose set can be designed as a medical disposable, which is discarded after the treatment.

The arterial blood line 201 and the venous blood line 202 optionally have an arterial clip 203 and a venous clip 204, respectively, with the aid of which the respective blood line can be closed.

The blood treatment appliance 100 comprises a pump device 101 for blood, which delivers the blood along the arterial blood line 201 in the direction of the gas exchange unit 300 and along the venous blood line 202 back to the patient. The pump device 101 for blood can be, for example, a diaphragm pump and preferably an occluding pump, for example a roller pump. On its return, the blood can flow through a venous blood chamber 205, which optionally has a venting device 206. The pump device 101 for blood can also be connected to a throttle or a valve for adjusting the flow.

The gas exchange unit 300 has a sweep gas inlet 301 and a sweep gas outlet 302. It can be designed as a gas exchanger, membrane oxygenator, CO2 removal system (e.g. multiECCO2R from the company EUROSETS S.r.l., Medolla (MO), Italy) or dialyser. The gas exchange unit 300 is separated (not shown here) by hollow fibre bundles, which together form a semipermeable membrane, into a region through which blood flows and a region through which sweep gas flows. In the gas exchange unit 300, there is removal of CO2 from the extracorporeal blood circuit 200 or delivery of O2 to the extracorporeal blood circuit 200, or both. The membrane can be coated with silicone or a silicone solution, with preferably the blood side of the hollow fibres being coated.

Downstream of the pump device 101 for blood, but upstream of the gas exchange unit 300, a feed point 207 for a fluid, for example a substituate solution, medicaments or an anticoagulant, can be provided. The anticoagulant is preferably a systemic anticoagulant, for example heparin, although it may also be a local anticoagulant, for example citrate.

Moreover, pressure sensors can be provided in the extracorporeal circuit 200, for example, as is indicated in the simplified view, an arterial pressure sensor PS1, which measures the pressure in the arterial blood line 201 upstream of the pump device 101 for blood, an arterial pressure sensor PS2, which measures the pressure in the arterial blood line 201 downstream of the pump device 101 for blood and upstream of the gas exchange unit 300, and a venous pressure sensor PS3, which measures the pressure in the venous blood line 202 downstream of the gas exchange unit 300.

In the venous blood line 202, downstream of the gas exchange unit 300, there is also a temperature sensor TS which measures the temperature of the blood as a treatment parameter. Alternatively or in addition, a temperature sensor can also be provided directly on the patient and, on the basis of the values measured there, a conclusion can be drawn concerning the temperature of the blood.

Furthermore, at least one sensor for measuring the blood flow rate in the extracorporeal circuit can be provided in a blood line 201, 202 upstream and/or downstream of the gas exchanger 300 or can be integrated (not shown here) in the pump device 101 for blood.

Besides the gas exchange unit 300, the blood treatment appliance 100 can also comprise one or more blood treatment units, for example a dialyser, an adsorber cartridge or a diagnostic unit (not shown here). In this way, several treatment techniques can be combined with one another.

The gas exchange unit 300 is, on the one hand, connected to the sweep gas circuit 400 via a sweep gas inlet 301 and a sweep gas outlet 302 and, on the other hand, connected to the extracorporeal blood circuit 200 via a blood inlet 303 and blood outlet 304.

The gas flows from a sweep gas source 401 via a first line section 402, for sweep gas delivery, into the gas exchange unit 300, and via a second line section 403, for sweep gas removal, out of the gas exchange unit 300.

The second line section 403, for sweep gas removal, can have a sensor PS4 for measuring the carbon dioxide partial pressure (pCO2) and/or for measuring the oxygen partial pressure (pO2). Alternatively or in addition, such a sensor can also be provided (not shown here) in the venous blood line 202 downstream of the gas exchange unit 300. However, instead of being measured by means of the sensor PS4, the pCO2 or pO2 can also be measured by blood gas analysis, in which a blood sample is taken from the extracorporeal circuit 200 at a removal site provided for this purpose.

The sweep gas circuit 400 can consist of a hose set which, for reasons of hygiene, can be designed as a medical disposable.

The sweep gas circuit 400 moreover has a device 404 for changing at least one sweep gas parameter. This device comprises a sweep gas pump 405 or, alternatively, a for example throttle-based flow regulator by which the sweep gas coming from the sweep gas source 401, which produces or supplies fresh sweep gas composed of room air, oxygen or a mixture of oxygen and other gases such as noble gases or nitrogen, is conveyed along the first line section 402 for sweep gas via the sweep gas inlet 301 into the gas exchange unit 300 and along the second line section 403 for sweep gas via the sweep gas outlet 302 out of the gas exchange unit 300. The sweep gas source 401 can supply the sweep gas in a container, for example a gas cylinder, or can be connected to a gas line from which sweep gas is continuously fed. When room air is used, the sweep gas source 401 can have or be connected to a particle filter, in order to supply sweep gas that is as far as possible free of harmful particles, for example dust.

Furthermore, the device 404 for changing at least one sweep gas parameter optionally comprises a heater 406 for heating the sweep gas (for example ProLUNG Meter from the company ESTOR, which heats the sweep gas and at the same time controls the sweep gas flow), and optionally a humidifier 407 (e.g. Bennet Cascade Humidifier from the company Robin Medical Ltd. or an Aircon respiratory air humidifier and heater from the company WILAmed) for humidifying the sweep gas.

Preferably upstream of the gas exchange unit 300, the sweep gas circuit 400 can have sensors (not shown here) for determining sweep gas parameters, for example the sweep gas temperature, sweep gas humidity and sweep gas flow rate.

The blood treatment appliance 100 moreover comprises a control unit 500 which is configured to regulate or control the device 404 for changing at least one sweep gas parameter. For this purpose, it can be connected for wired or wireless signal transmission to each of the aforementioned components, in particular to the pump device 101 for blood, to the temperature sensor TS, to the sensor PS1 and/or PS2 for measuring the pCO2 and/or pO2, to the sweep gas source 401, and to the individual components of the device 404 for changing at least one sweep gas parameter.

The control unit 500 is designed to operate the sweep gas pump 405 with electrical powder in order to regulate the flow rate of the sweep gas. The control unit 500 is moreover designed to operate the heater 406 and humidifier 407 with electrical power in order to regulate the temperature and the humidity of the sweep gas.

By means of the appliance electronics, the power to the sweep gas pump 405, the heater 406 or the humidifier 407 can be controlled, regulated, stored and/or indicated in accordance with one or more values detected by the one or more sensors.

The method according to the invention can thus be carried out largely or completely by the control unit 500. In particular, the control unit can perform those steps that do not require human assistance and/or intervention.

In a further embodiment, the blood treatment appliance 100 can additionally comprise a mechanical ventilation unit 600, with which a CO2 and O2 exchange can likewise be carried out. Mechanical ventilation (shown only schematically in FIG. 1) can either take place non-invasively by means of a helmet or a mask 601 or invasively by means of an endotracheal tube 602 which is inserted through the nose or mouth or via a tracheal cannula which is inserted through an artificial opening into trachea. The control unit 500 is also connected to the mechanical ventilation unit 600 and is configured such that it controls the latter in accordance with the treatment parameters and with the measured values from the sensors.

In the method for controlling the device 404 for changing at least one sweep gas parameter and/or for controlling the mechanical ventilation unit 600, values at least for the treatment parameters, for example the patient's temperature and/or the pCO2 and/or the flow rate, are initially detected by the sensors (actual values) and transmitted to the control unit 500.

The control unit 500 compares the detected values against the values (setpoint values), which have been input by the user and/or preset, and calculates manipulated variables for the device for changing at least one sweep gas parameter.

Based on the manipulated variables that have been calculated, the control unit 500 controls the sweep gas pump 405 and/or the heater 406 and/or the humidifier 407 in such a way that the flow rate of the sweep gas is increased or reduced and/or the sweep gas is heated and/or humidified. The sweep gas pump 405, the heater 406 and the humidifier 407 can be activated or deactivated independently of one another.

The method according to the invention is set forth in more detail below on the basis of exemplary embodiments.

As was mentioned at the outset, the flow rate, the temperature and the humidity of the sweep gas used during ECLS treatment are factors which influence both the CO2 removal rate and the heat loss of the blood.

For example, a high flow rate of sweep gas results in a high level of CO2 removal. However, the heat loss also increases along with the rising flow rate of sweep gas, and the heat loss even undergoes a proportional increase.

It is therefore desirable to control the treatment parameters in such a way that they are adapted to the state of the patient and to the treatment status and are set optimally for the treatment goal.

FIG. 2 shows an example of a CO2 removal curve (carbon dioxide transfer rate (CTR)) according to the sweep gas flow, i.e. the CO2 removal from the blood, at a constant blood flow rate and at varying sweep gas rates, which were determined in a test arrangement in the laboratory. Such CO2 removal curves are typical for specific gas exchange units.

At a ratio of sweep gas flow to blood flow of 15:1, i.e. for example a blood flow of 0.5 l/min and a maximum sweep gas flow of 7.5 l/min, the CO2 removal rate is at its greatest. For the treatment goal of maximum CO2 removal, the setting with a ratio of sweep gas flow to blood flow of 15:1 is optimal.

If the sweep gas flow rate is reduced and thus at the same time the ratio of sweep gas flow to blood flow is changed, the heat loss decreases, but also the quantity of CO2 removed.

At a ratio of sweep gas flow to blood flow of 5:1, i.e. for example a blood flow of 0.5 l/min and a sweep gas flow of 2.5 l/min, the CO2 removal rate decreases by only 10%. Upon a further decrease of the sweep gas flow, the curve becomes steeper, which means that the CO2 removal rate drops more strongly. Therefore, for the treatment goal of minimum heat loss, the optimal setting is a ratio of sweep gas flow to blood flow of 5:1.

As has already been described above, the sweep gas temperature and humidity also have an influence on the level of heat loss of the patient.

FIG. 3 shows by way of example the drop in the carbon dioxide partial pressure ΔpCO2 in mmHg between blood inlet 303 and blood outlet 304 of the gas exchanger 300 under different conditions of the sweep gas over time in seconds.

It will be seen from the figure that cold and at the same time dry sweep gas (shown in the figure as a dotted line) has by comparison the highest CO2 removal rate.

However, cold and dry sweep gas also causes the greatest heat loss. By heating and humidifying of the sweep gas, the heat loss decreases, while at the same time the CO2 removal rate falls.

However, the CO2 removal rate does not decrease proportionally: by heating of the sweep gas (shown in the figure as a broken line), the removal rate in the example shown drops by ca. 10%; by heating and humidifying (shown in the figure as a continuous line), it drops a further 10% at most. In view of these circumstances, a setting optimal for the condition and treatment status of the patient can be found for the treatment goal.

In a further embodiment, a calculation and/or balancing of the total amount of removed CO2 can be effected based on the detected pCO2.

The calculation can take place for example by multiplying the CO2 concentration in the outflowing sweep gas (e.g. in %) by the sweep gas flow rate (ml/min).

Commercially available pCO2 sensors also already take account of the humidity of the measured air. It is assumed that the CO2 concentration of the sweep gas is equal to zero, or at least known and near zero, like air.

In a further embodiment, the total CO2 removal rate can be calculated by the control device while mechanical ventilation takes place at the same time via an electronic interface. The calculation is made by addition of the removal rate of the natural lungs and the removal rate of the membrane lungs.

By means of an additional CO2 sensor at the outlet of the non-invasive (mask) ventilation unit, it is possible, together with the sweep gas flow rate (CO2 fraction in the sweep gas multiplied by the flow rate of the sweep gas), to form an overall balance for removed CO2 analogously to a mechanical ventilation appliance. The two removal rates determined (natural lungs and membrane lung) are added together.

FIG. 4 shows a schematic view of the blood treatment appliance 100 according to the invention.

What has been stated concerning the individual components in FIG. 1 also applies to FIG. 4. Therefore, only those features that appear in FIG. 4 and not in FIG. 1 are discussed below.

Thus, FIG. 4 shows the aforementioned control unit 500, which is connected to an extracorporeal blood treatment appliance 200, a gas exchange unit 300, a device 404 for changing the sweep gas parameters, and a gas source 401. The blood treatment appliance 200 can be connected to a patient P via a vascular access.

FIG. 4 further shows that the control unit 500 is connected to the hardware and software unit 700, which comprises further components. The data exchange between the individual components of the hardware and software unit 700 takes place via corresponding interfaces.

For example, the hardware and software unit 700 can have a user interface 701.

The user interface 701 can be designed, for example, as a graphic user interface. By way of a keyboard, a mouse or a touch screen, the user is able to select or modify a treatment goal.

According to the invention, the treatment goal is selected in view of the contrary objectives of maximizing the CO2 removal and minimizing the heat loss at different settings of the sweep gas parameters.

Based on what is specified by the user B, the control unit 500 carries out predetermined procedures individually or in combination. An entry made on the user interface 701 by the user can, for example, trigger a change in the sweep gas temperature, the sweep gas humidity and the sweep gas flow rate, individually or in combination.

In this way, depending on the goal specified by the user via control of the device 404 for changing at least one sweep gas parameter, the blood treatment appliance 100 can be set to the respective treatment goal on the basis of the input treatment goal and in accordance with the at least two treatment parameters pCO2 and/or blood temperature and/or blood flow rate.

Alternatively or in addition, the goal can also be specified in an automated manner by a script 704 or by the automated execution of various predetermined steps.

Alternatively or in addition, the goal can also be specified using a data-based decision support system 702 which takes into consideration data from at least one data source 703, for example a hospital data system, and which proposes these to the user B for selection for the specified goal. The data can be, for example, a physiological parameter or a setpoint value for a sweep gas or treatment parameter.

After this specified goal has been confirmed or modified, the data are transmitted to the control unit 500. The latter triggers an in each case different combination of sweep gas flow rate and/or sweep gas temperature and/or sweep gas humidity.

At least 33 combinations are conceivable, even considerably more if using individual gradations of heating, humidification and flow rate.

Some combinations may occur more frequently and others less often. For example, on account of the greater outlay, humidification will generally be effected only in combination with heating of the sweep gas. Conversely, although heating without humidification can contribute to a reduction of the temperature loss, according to the enthalpy of gases the heating alone of a gas leads only to a slight increase of the specific thermal content. An additional humidification abruptly increases the enthalpy.

For example, when an initially dry sweep gas is heated from 20° Celsius (10% relative humidity) to 37° Celsius (5% relative humidity), the specific thermal content increases from 23.8 kJ per kg dry air to 42.2 kJ per kg dry air. If the sweep gas is humidified concurrently with the heating, the specific thermal content increases to 142.8 kJ per kg dry air.

This clearly illustrates that the greater proportion of the heating is effected by humidification of the sweep gas before the gas exchanger, since the latter crucially decreases the vapour pressure difference across the membrane as driving force for the passage of the water vapour, which reduces the amount of water evaporating on the membrane and thus reduces the thermal loss (evaporative cooling).

FIG. 5 shows examples of measured values for the blood temperature in degrees Celsius at the blood inlet 303 and blood outlet 304 for different sweep gas temperatures and humidity, over the duration of treatment, indicated in hours.

The blood temperature over a certain time span without an extracorporeal treatment is shown by a solid black line. This temperature is relatively constant at 36.5 °C.

The blood temperature over a certain time span during an extracorporeal treatment, without use of a sweep gas, is shown by a continuous grey line. There is a discernible heat loss of approximately 0.5 °C, which arises through heat radiation and conduction across the surface of the extracorporeal blood hose system.

The blood temperature over a certain time span during an ECLS, with use of a sweep gas at room temperature, i.e. without additional heating and humidification, is shown by a broken grey line. It will be seen that the blood temperature at the start of the treatment is at a comparatively low level of around 35.7 °C and drops further during the treatment, until it settles at 35.5 °C.

The blood temperature over a certain time span during an ECLS, with use of sweep gas which is only heated and not humidified, is shown by a broken black line. It will be seen that the blood temperature at the start of the treatment is at a very low level of 35.4 °C, after which it rises and settles at a constant temperature level of 35.5 °C.

The blood temperature over a certain time span during an ECLS, with use of heated and humidified sweep gas, is shown by a thin black line. At the start of the treatment, the temperature is 36 °C and is therefore slightly low, but it does not drop any further.

In summary, it will be seen that warm and humidified sweep gas causes a similarly small temperature drop like eliminated sweep gas. The influence that the use of sweep gas has on the temperature, in the sense of a decrease, can thus be reduced to almost zero by heating and humidifying.

Overall, it may be said that controlling the humidity of the sweep gas has a particular relevance in reducing the heat loss.

It is conceivable for the user to change the specified goal during the course of treatment. For example, at the start of treatment, it may be expedient to obtain a rapid drop of the pCO2 in the blood for a certain time interval. Thereafter, for a longer time interval, the main concern is then to avoid the heat loss. Intermediate goals are also conceivable. The user can input the modified goal specification via the user interface.

A script with established sequences of treatment steps, e.g. rapid initial lowering of patient pCO2 and subsequent moderate CO2 removal with simultaneous compensation of the heat loss by heating and humidifying the sweep gas, can serve to control the appliance. In treatment situations where no humidification and heating are available, the sweep gas flow can also be reduced instead.

### LIST OF REFERENCE SIGNS

100 blood treatment appliance
101 pump device for blood
200 extracorporeal blood circuit
201 arterial blood line
202 venous blood line
203 arterial clip
204 venous clip
205 venous blood chamber
206 venting device
207 feed point for a fluid
300 gas exchange unit
301 sweep gas inlet
302 sweep gas outlet
303 blood inlet
304 blood outlet
400 sweep gas circuit
401 sweep gas source
402 first line section, for sweep gas delivery
403 second line section, for sweep gas removal
404 device for changing at least one sweep gas parameter
405 sweep gas pump
406 heater
407 humidifier
500 control unit
600 mechanical ventilation unit
601 mask
602 tube
700 hardware and software unit
701 user interface
702 decision support system
703 data source
704 script
B user
P patient
PS1,PS2 arterial pressure sensor
PS3 venous pressure sensor
PS4 sensor for measuring pCO2
TS temperature sensor

1. A blood treatment appliance for gas exchange via a gas exchange unit having two regions separated by a membrane, wherein one region has blood flowing through it and the other region has sweep gas flowing through it, comprising:
a pump device for blood,
sensors for measuring at least two treatment parameters,
a device for changing at least one sweep gas parameter,
and a control unit which is connected to the pump device for blood, to the sensors for measuring at least two treatment parameters and to the device for changing at least one sweep gas parameter,
wherein
the control unit is configured in such a way that it controls the device for changing at least one sweep gas parameter in accordance with the at least two treatment parameters.

2. The blood treatment appliance according to claim 1, wherein the gas exchange is a removal of carbon dioxide from the extracorporeal blood circuit.

3. The blood treatment appliance according to claim 1, wherein the gas exchange unit is a membrane oxygenator or a dialyser.

4. The blood treatment appliance according to claim 1, wherein the at least two treatment parameters are the patient's temperature and or the carbon dioxide partial pressure and/or the blood flow rate.

5. The blood treatment appliance according to claim 1, wherein one the patient's temperature is a treatment parameter measured in the extracorporeal circuit or a treatment parameter measured on the body of the patient, and in that the carbon dioxide partial pressure is a treatment parameter measured in the sweep gas or in the extracorporeal circuit.

6. The blood treatment appliance according to claim 1, wherein one at least one sweep gas parameter is the temperature and/or the humidity and/or the flow rate of the sweep gas.

7. The blood treatment appliance according to claim 1. wherein the device for changing at least one sweep gas parameter comprises
a sweep gas pump or flow regulator for delivering the sweep gas, and/or
a heater for heating the sweep gas, and/or
a humidifier for humidifying the sweep gas.

8. The blood treatment appliance according to claim 1, wherein the control unit is connected to a user interface for input of at least one treatment goal.

9. The blood treatment appliance according to claim 1, wherein the control unit is connected to a decision support system

10. The blood treatment appliance according to claim 1, wherein one it is connected via a hose system to the patient and the gas exchange unit or to the patient and the gas exchange unit and at least one further treatment unit.

11. The blood treatment appliance according to claim 1, wherein the blood treatment appliance additionally comprises a mechanical ventilation unit and in that the control unit is configured in such a way that it controls the device for changing at least one sweep gas parameter and/or controls the mechanical ventilation unit in accordance with the at least two treatment parameters.

12. A method for controlling and/or regulating the device for changing at least one sweep gas parameter and/or the mechanical ventilation unit in the blood treatment appliance according to claim 1, said method comprising the following steps:
detecting at least two treatment parameters by means of sensors,
comparing the detected treatment parameters against the treatment parameters input by the user and/or preset,
calculating manipulated variables on the basis of the comparison,
controlling the sweep gas pump and/or the heater and/or the humidifier on the basis of the calculated manipulated variables.

13. The method according to claim 12, wherein a calculation and/or balancing of the total quantity of the carbon dioxide removed by the appliance is effected on the basis of the value for the carbon dioxide partial pressure and for the sweep gas flow.

14. The blood treatment appliance according to claim 1, wherein the blood treatment appliance is connected via a hose system to the patient and the gas exchange unit or to the patient and the gas exchange unit and at least one further treatment unit that is a dialyser or an adsorber cartridge.
